(19) United States
(12) Patent Application Publication
(10) Pub. No.: US 2024/0344098 A1
(43) Pub. Date: Oct. 17, 2024

(54) METHODS AND SYSTEMS FOR CAPPING NUCLEIC ACID MOLECULES

(71) Applicant: Quantoom Biosciences S.A., Nivelles (BE)

(72) Inventors: Kristof VANDEKERCKHOVE, Humbeek (BE); Grégory GODEFROI, Louvain-la-Neuve (BE); Blandine DAVID, Floreffe (BE)

(21) Appl. No.: 18/750,619

(22) Filed: Jun. 21, 2024

Related U.S. Application Data
(63) Continuation of application No. PCT/EP2022/087738, filed on Dec. 23, 2022.
(60) Provisional application No. 63/293,539, filed on Dec. 23, 2021.

(30) Foreign Application Priority Data
Jan. 20, 2022 (BE) BE2022/05036

(51) Int. Cl. C12P 19/34

(52) U.S. Cl. CPC C12P 19/34

(57) ABSTRACT

Described herein are systems for capping nucleic acid. Also described herein are methods for capping nucleic acid with the systems described herein.

### CROSS-REFERENCE

This application is a continuation of International Application No. PCT/EP2022/087738, filed Dec. 23, 2022, which claims priority to U.S. Provisional Patent Application No. 63/293,539, filed Dec. 23, 2021, and Belgium Application No. BE2022/5036, filed Jan. 20, 2021, each of which is incorporated herein by reference in its entirety.

### BACKGROUND

In vitro nucleic acid processing is widely used in biomedical or bioscience fields. One of the in vitro nucleic acid processing or manufacturing methods involves capping of messenger RNA (mRNA) for manufacturing of mRNA or peptide encoded by the mRNA in industrial quantities. Two main strategies are currently used for production of 5′-capped mRNA: co-transcriptional capping, whereby a synthetic oligonucleotide integrating the cap structure is incorporated during transcription of the template DNA strand; and post-transcriptional capping, whereby the biosynthesis of the cap structure and associated reactions is enzymatically catalyzed.

Co-transcriptional capping is described by Whitley et al., 2021. In post-transcriptional capping, the capping reaction is inhibited by the by-products of IVT and thus prior to the capping the mRNA is usually purified. WO201815714, WO2020041793 and Fuchs et al., 2016, describe mRNA purification methods prior to capping. These purification steps are usually time-consuming and can result in loss of a significant part of the sample.

The current invention aims to develop a simplified post-transcriptional capping method, that overcomes at least some of the above mention drawbacks.

### SUMMARY

Efficient post-transcriptional capping of mRNA requires prior treatment of the reaction harvest obtained after in vitro transcription (IVT). The traditional method of pre-treatment involves at least one purification operation between the IVT step and the enzymatic capping step in order to ensure sufficient capping efficiency. Such steps increase process duration and complexity and decrease the overall capped RNA molecule yield. Accordingly, systems and methods for simple, inexpensive and fast treatment of mRNA reaction harvest to ensure efficient enzymatic capping downstream are of interest.

Described herein, in some aspects, is a method for producing at least one capped ribonucleic acid (RNA) molecule, comprising: providing a plurality of uncapped RNA molecules in a first solution; removing a plurality of molecules that has a molecular weight of at most about 1000 kDa in the first solution to form a second solution, so that a post-transcriptional capping efficiency of at least 75% is achieved; contacting the second solution with a plurality of capping enzyme molecules; and adding a cap structure to a 5′ end of an uncapped RNA molecule to form the at least one capped RNA molecule. In some embodiments, the plurality of molecules has a molecular weight of at most about 800 kDa. In some embodiments, the plurality of molecules has a molecular weight of at most about 600 kDa. In some embodiments, the plurality of molecules has a molecular weight of at most about 500 kDa. In some embodiments, the plurality of molecules has a molecular weight of at most about 400 kDa. In some embodiments, the plurality of molecules has a molecular weight of at most about 200 kDa. In some embodiments, the plurality of molecules has a molecular weight of at most about 100 kDa. In some embodiments, the plurality of molecules has a molecular weight of at most about 30 kDa. In some embodiments, the plurality of molecules has a molecular weight of at most about 10 kDa. In some embodiments, the plurality of molecules has a molecular weight of at most about 5 kDa. In some embodiments, the plurality of molecules has a molecular weight of at most about 3 kDa. In some embodiments the removing the plurality of molecules comprises filtration of the first solution against a filter, wherein the filter comprises a nominal pore size measured in a molecular weight cut off (MWCO) of about 800 kDa, 600 kDa, 500 kDa, 400 kDa, 200 kDa, 100 kDA, 50 kDA 30 kDa, 10 kDa, 5 kDa, 3 kDa, or 1 kDa. In some embodiments, the filtration is by continuous or discontinuous diafiltration. In some embodiments, the filtration comprises tangential flow filtration. In some embodiments, the removing the plurality of molecules comprises conducting a dialysis of the first solution in a suitable medium. In some embodiments, the removing the plurality of molecules does not comprise an additional purification step. In some embodiments, the additional purification step is performed by a chromatography. In some embodiments, the plurality of uncapped RNA molecules is generated via an in vitro transcription (IVT) reaction. In some embodiments, the plurality of capping enzyme is selected from the group consisting of Cap-specific mRNA (nucleoside-2′-O—)-methyltransferase, Vaccinia capping enzyme (VCE), Bluetongue Virus capping enzyme, Chlorella Virus capping enzyme, S. cerevisiae capping enzyme, Mimivirus capping enzyme, African swine fever virus capping enzyme, and Avian Reovirus capping enzyme. In some embodiments, adding the cap structure to the 5′ end of the uncapped RNA molecule occurs at an efficiency of at least 70%. In some embodiments, adding the cap structure to the 5′ end of the uncapped RNA molecule occurs at an efficiency of at least 75%. In some embodiments, adding the cap structure to the 5′ end of the uncapped RNA molecule occurs at an efficiency of at least 77%. In some embodiments, adding the cap structure to the 5′ end of the uncapped RNA molecule occurs at an efficiency of at least 80%. In some embodiments, adding the cap structure to the 5′ end of the uncapped RNA molecule occurs at an efficiency of at least 85%. In some embodiments, adding the cap structure to the 5′ end of the uncapped RNA molecule occurs at an efficiency of at least 90%. In some embodiments, adding the cap structure to the 5′ end of the uncapped RNA molecule occurs at an efficiency of at least 95%. In some embodiments, adding the cap structure to the 5′ end of the uncapped RNA molecule occurs at an efficiency of at least 97%. In some embodiments, adding the cap structure to the 5′ end of the uncapped RNA molecule occurs at an efficiency of at least 98%. In some embodiments, adding the cap structure to the 5′ end of the uncapped RNA molecule occurs at an efficiency of at least 99%. In some embodiments, adding the cap structure to the 5′ end of the uncapped RNA molecule occurs at an efficiency of at 100%. In some embodiments, a concentration of the plurality of molecules is reduced by at least 50%, 60%, 70%, or 80%, or 85%, or 90%, or 95%, or 96%, or 97%, or 98%, 99%, or approaching 100% in the second solution compared to a concentration of the plurality of molecules in the first solution. In some embodiments, a concentration of the plurality of molecules is reduced by at least 99.9975% in the second solution compared to a concentration of the plurality of molecules in the first solution. In some embodiments, the concentration of the plurality of molecules in the second solution is less than or equal to 50%, or 40%, or 30%, or 20%, or 15%, or 10%, or 5%, or 4%, or 3%, or 2%, or 1% of the concentration of the plurality of molecules in the first solution. In some embodiments, the method further comprises synthesizing a peptide or protein utilizing the at least one capped RNA molecule.

Described herein, in some aspects, is a pharmaceutical composition obtained using a method described herein. In some embodiments, the pharmaceutical composition is a vaccine.

Described herein, in some aspects, is a peptide or protein obtained using a method described herein. In some embodiments, the peptide or protein is produced in vivo. In some embodiments, the peptide or protein is produced in vitro. In some embodiments, the peptide or protein is a prophylactic or a therapeutic peptide or protein.

Described herein, in some aspects, is a composition comprising a plurality of 5′ capped RNA molecules, wherein said RNA molecules are obtained by means of an in vitro transcription reaction and wherein said capping occurred post-transcriptionally, said composition comprises reagents for in vitro transcription, and wherein the concentration of RNA in said composition is less than 20 mg/ml.

Described herein, in some aspects, is a composition comprising a plurality of 5′-capped RNA molecules, said RNA molecules are obtained by means of an in vitro transcription reaction and wherein said capping occurred post-transcriptionally, wherein the capping reaction efficiency is at least 75% without utilizing chromatography.

Described herein, in some aspects, is a composition comprising a plurality of 5′-capped and uncapped RNA molecules, said RNA molecules are obtained by means of an in vitro transcription reaction and wherein said capping occurred post-transcriptionally, wherein a ratio between the plurality of uncapped RNA molecules and the plurality of capped RNA molecules is between 0.0001 to 0.3.

Described herein, in some aspects, is a system comprising: at least one container containing the first solution or the second solution of any one of previous claims; and at least one filter for separating the plurality of molecules of any one of previous claims.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates a non-limiting example of an experimental protocol comprising buffer exchange (e.g., diafiltration) for removing the plurality of molecules that inhibits capping reaction from the first solution containing the uncapped RNA molecules.

FIG. 2 illustrates another non-limiting example of an experimental protocol comprising dialysis for removing g the plurality of molecules that inhibits capping reaction from the first solution containing the uncapped RNA molecules.

FIG. 3 illustrates a non-limiting example of a system described herein, where the system comprises at least one container (301) comprising a first solution (302) comprising at least one uncapped nucleic acid. The first solution (302) may be contacted with a membrane filter (303). The contacting with the membrane filter (303) removes a plurality of molecules capable of permeating the membrane that inhibits capping reaction, thus allowing capping reaction to occur to generate capped RNA molecules (304). The capped nucleic acid (304) may be further purified or filtered to increase the concentration (305) of the capped RNA molecules (304) and to further remove undesired substances contained in the solution.

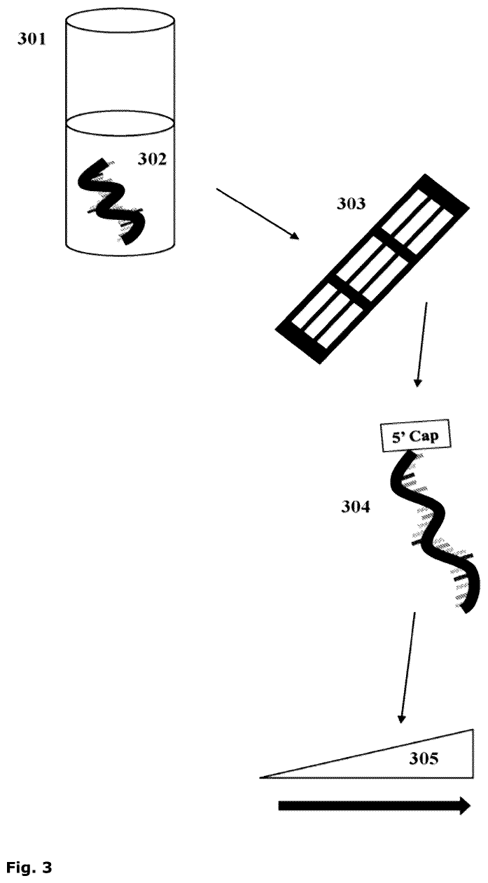

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Efficient post-transcriptional capping of messenger RNA produced by in vitro transcription (IVT) is only possible after an intermediate treatment of the IVT reaction product. This intermediate treatment typically involves a purification operation (such as chromatography), usually in combination with a tangential flow filtration step. This suggests that the in vitro transcription reaction product contains substances that interfere with the capping reagents. Enzymatic capping immediately following IVT, without intermediate treatment of the reaction product, produces small amount or close to 0% capped mRNA molecules. When adding the capping reagents to the IVT for “concurrent transcription and capping” (one-pot reaction), marginal capping may be achieved (less than 10%). This confirms that successful capping requires some intermediate treatment of the IVT product. A conventional intermediate treatment, as mentioned above, involves one or several purification steps, which comes with several disadvantages: inevitable loss of product (decreased process yield); increased process complexity requiring development of extra steps; the need for additional equipment; and increased cost due to product loss and the cost of the extra operation steps. The need for additional equipment also leads to increased operational cost, increased system footprint, and extended process duration.

Described herein are systems and methods for removing (by filtering or dialyzing) the plurality of molecules that inhibits capping reaction based on size, where the plurality of molecules is smaller and may be filtered out through a nominal pore size below the molecular size of RNA. In some embodiments, the systems and methods comprise direct filtration through a filter (e.g., a membrane filter) followed by re-solubilization of the retentate in a suitable solvent (e.g. water); diafiltration against a suitable number of diavolumes of a suitable solvent; or dialysis of the IVT harvest in a suitable medium to remove substances that inhibit the capping reaction of adding cap structures to uncapped RNA molecules. In some cases, the systems and methods may comprise dilution of the IVT RNA molecules followed by ultrafiltration to remove a portion of the solvent. In some embodiments, the systems and methods lead to incomplete removal of the solutes, depending on the dilution factor chosen. For example: a 50× dilution followed by reconcentration to the original solvent volume removes 98% of the solutes.

In some embodiments, the systems and methods described herein allow capping reaction to occur without the need to first isolate or purify the uncapped RNA using conventional methods, such as chromatography. In some embodiments, the systems and methods described herein achieves 5′ capping efficiency of mRNA in vitro that is substantially similar to the capping efficiency achieved by using conventional uncapped RNA molecule purification methods. In some embodiments, the systems and methods described herein comprises contacting a solution comprising uncapped RNA molecules with a filter. In some cases, the filter material comprises ceramic. In some cases, the filter material comprises one or more minerals. In some case, the filter material comprises one or more metals. In some cases, the filter material comprises a polymer. The filter comprises pores with a pore size that: allows passing through of the plurality of molecules that inhibits the capping reaction but retains the uncapped RNA molecules. By performing such medium exchange, the plurality of molecules that inhibit capping reaction of adding cap structures to uncapped RNA molecules is removed from the solution comprising the uncapped RNA molecules until the concentration of the plurality of molecules that inhibit capping reaction is reduced to a level that no longer inhibit capping reaction. In some embodiments, the solution after buffer exchange is contacted with a plurality of capping enzymes and other reagents to form capped RNA molecules. In some embodiments, the capped RNA molecules obtained from the systems and methods described herein may be utilized for purposes such as manufacturing of pharmaceuticals or diagnostic compositions.

### Systems

Described herein are systems for processing nucleic acids with the methods described below, such as processing solutions containing uncapped RNA molecules derived from in vitro transcription (IVT) reactions to avoid inhibition of capping reactions related to adding a cap structure to an uncapped RNA molecule. The system may comprise an upstream portion directed to provide IVT reaction mixtures containing the uncapped RNA molecules. In some cases, the system may comprise a downstream portion for further processing of capped RNA molecules, such as purification to remove undesired substances and tangential flow filtration to change the composition and the concentration of the solution of capped RNA molecules. In some embodiments, the system may be further configured to manufacture compounds, biomolecules, or pharmaceutical compositions using the capped RNA as input. For example, the system described herein may synthesize or increase yield of synthesizing an antigen encoded by the capped RNA or capped mRNA, where the antigen may be further formulated into a vaccine. In some embodiments, the system comprises components or devices for initiating or maintaining biological reactions. In some embodiments, the system may be configured to effect any sort of appropriate process. Non-limiting examples of processes to which system disclosed herein may be suited to include production of a biological compound; production of a pharmaceutical or biopharmaceutical compound; RNA synthesis, including IVT and post-transcriptional processes and RNA purification; protein synthesis, including cell-dependent protein synthesis and cell-free protein synthesis (CFPS); or a combination thereof.

In some embodiments, the system described herein is modular, where each component of the system may be independently assembled or disassembled based on the functionality needed. In some embodiments, the system comprises a continuous reactor or a batch reactor. In some cases, the system may comprise a continuous reactor. The system may be operated in a continuous mode. In other cases, the system may comprise a batch reactor. The system may be operated in a non-continuous mode or a batch reaction mode. In other cases, the system may comprise a combination of a continuous reactor and a batch reactor and the system may be operated in a semi-continuous mode.

In some cases, a system as disclosed herein may comprise more than one container. The more than one containers may be in fluid communication with one another, some subset of the more than one containers may be in fluid communication with the same or another subset of the more than one containers, or the more than one containers may not be in fluid communication. The system may be programmable to transport the medium from one container to another after a certain time period. In some cases, the time period may be determined by an incubation or reaction time of a reagent or component of the medium, the length of the container, the volume of the container, the flow rate of the medium through the container, or some combination thereof. In some embodiments, the system described herein comprises at least one filter described herein. In some embodiments, the filter (e.g., a membrane filter) can be positioned between two reactors for generating the second solution from the first solution.

In some embodiments, the system described herein comprises at least one container. A first container holds a first solution comprising the uncapped RNA molecules. In some embodiments, the first solution may be filtered for obtaining a second solution. The filtration units may comprise a dead-end filtration unit, spin filtration unit, a tangential flow filtration (TFF) unit, an alternating tangential flow (ATF) filtration unit, or any other suitable filtration unit known in the art. In some embodiments, the filtration unit is in fluid communication with the first container. In some embodiments, the filtration unit is not in fluid communication with the first container. After the filtration process, in some cases, the systems may further comprise a mixing unit. The filtered second solution may go through the mixing device. In some embodiments, the filtered second solution may not go through the mixing device. In some embodiments, the mixing device is contained within the filtration unit.

In some embodiments, the filtered second solution may be transferred to a second container for capping reaction to occur. In some cases, the capping reaction of adding a cap structure to an uncapped RNA molecule is conducted in the second container. In some embodiments, the filtration unit is in fluid communication with the second container. In some embodiments, the filtration unit is not in fluid communication with the second container. Any necessary reagents, such as capping enzymes as described herein, capping enzyme substrates, buffers and salts, methyl donor, and other reagents may be added to the second container via a valve or an opening. In some embodiments, the filtered second solution may be transferred back to the first container for capping reaction to occur. In some cases, the capping reaction of adding a cap structure to an uncapped RNA molecule is conducted in the first container. Any necessary reagents, such as capping enzymes as described herein, capping enzyme substrates, buffers and salts, methyl donor, and other reagents may be added to the first container via a valve or an opening.

In some embodiments, the system described herein comprises at least one container. A first container holds a first solution comprising the uncapped RNA molecules. In some embodiments, the first solution may undergo a dialysis process for obtaining a second solution. In some embodiments, the system comprises a dialysis device, which comprises a suitable filter for performing dialysis process. In some embodiments, the dialysis device is in fluid communication with the first container. In some embodiments, the dialysis device is not in fluid communication with the first container. After dialysis process, in some cases, the systems may further comprise a mixing unit. The treated second solution may go through the mixing device. In some embodiments, the treated second solution may not go through the mixing device. In some embodiments, the mixing device is contained within the dialysis unit.

After dialysis or dialysis plus mixing, the treated second solution is transferred to a second container for conducting capping reaction. In some cases, the capping reaction of adding a cap structure to an uncapped RNA molecule is conducted in the second container. In some embodiments, the dialysis unit is in fluid communication with the second container. In some embodiments, the dialysis unit is not in fluid communication with the second container. Any necessary reagents, such as capping enzymes as described herein, capping enzyme substrates, buffers and salts, methyl donor, and other reagents may be added to the second container via a valve or an opening. In some embodiments, the filtrated second solution may be transferred back to the first container for capping reaction to occur. In some cases, the capping reaction of adding a cap structure to an uncapped RNA molecule is conducted in the first container. Any necessary reagents, such as capping enzymes as described herein, capping enzyme substrates, buffers and salts, methyl donor, and other reagents may be added to the first container via a valve or an opening.

The solution may be transported from one part of the system to another (e.g., from one segment to another) or into or out of the system by the opening or closing of valves. Valves may be directed to open or close at certain times by the system. The system may further comprise pumps or other means, which are additionally directed by the system, for transporting the solution. n some embodiments, the system comprises a purification component or device for capturing the compound or biomolecule synthesized or present in the solution (e.g., the capped RNA molecule or polypeptide encoded from the capped RNA molecule) to remove unwanted substances. Non-limiting example of the purification component or device includes chromatography or filtration.

### Methods

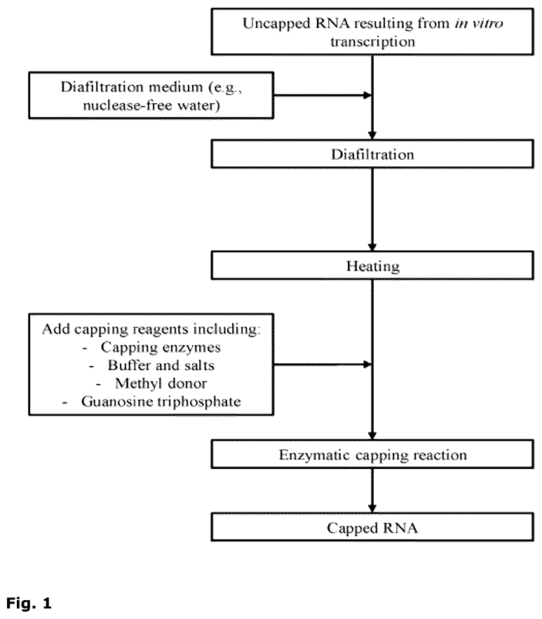

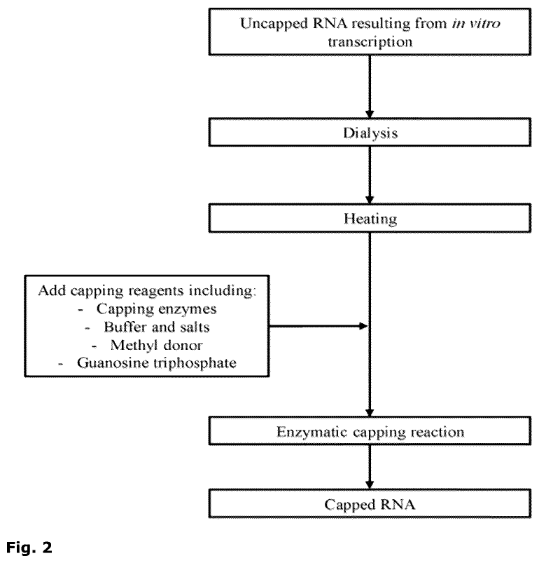

Described herein are methods for capping RNA molecules synthesized from IVT reaction. In some embodiments, the method utilizes the systems described herein for removing and separating the plurality of molecules that inhibits the capping reaction from the uncapped RNA molecules or uncapped mRNA molecules. In some embodiments, the method comprises obtaining a first solution comprising the uncapped RNA molecules. In some embodiments, as illustrated in FIG. 1, the first solution is contacted with at least one filter (e.g., a membrane filter) to form a second solution, where the plurality of molecules that inhibits the capping reaction is passed through the pore of the filter and separated from the uncapped RNA molecules or uncapped mRNA molecules. In some embodiments, the filtering of the first solution by contacting with the filter decreases a concentration of the plurality of molecules that inhibits capping reaction in the second solution. In some embodiments, as illustrated in FIG. 2, the first solution undergoes dialysis for buffer exchange, where the plurality of molecules may pass through the pores of the dialysis filter (e.g., dialysis membrane filter). In some embodiments, the dialysis filter comprises the same material or pore size as the at least one membrane filter described herein. In some embodiments, the plurality of molecules in the second solution is removed or decreased by at least 50%, 60%, 70%, 80%, 85%, 90%, 95%, 96%, 97%, 98%, 99%, or any percentages in between the aforementioned percentage by the filtering or dialysis of the filter. In some embodiments, the plurality of molecules in the second solution is less than or equal to 50%, 40%, 30%, 20%, 15%, 10%, 5%, 4%, 3%, 2%, 1%, or less of the concentration of the plurality of molecules in the first solution.

In some embodiments, the filter removes and separates a plurality of molecules that inhibits or interferes with capping reaction of adding cap structures to uncapped RNA molecules in the first solution to obtain the second solution, where the filter separates the plurality of molecules comprising a molecule weight cut off (MWCO) of at most about 1 kDa, 3 kDa, 5 kDa, 10 kDa, 30 kDa, 50 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kD a, 950 kDa 1000 kDa, or any molecular weight between the aforementioned molecular weight values.

With respect to filtration/diafiltration/ultrafiltration process, in some embodiments, an optional dilution step of the IVT reaction mixture, i.e, the first solution containing a plurality of uncapped RNA molecules is performed before the filtration process. The diluent may be purified water or any other suitable solution that does not interfere with any downstream reactions. In some embodiments, the IVT reaction mixture is diluted by at least 1-fold, at least 2-fold, at least 3-fold, at least 4-fold, at least 5-fold, at least 6-fold, at least 7-fold, at least 8-fold, at least 9-fold, at least 10-fold, at least 12-fold, at least 12-fold, at least 14-fold, at least 16-fold, at least 18-fold, at least 20-fold, at least 30-fold, at least 40-fold, at least 50-fold, at least 60-fold, at least 70-fold, at least 80-fold, at least 90-fold, at least 100-fold, at least 1000-fold, at least 10000-fold, at least 20000-fold, at least 30000-fold, at least 40000-fold or any numerical numbers in between the aforementioned dilution factors to form a second solution, where the capping efficiency in the second solution is increased compared to the capping efficiency of the undiluted solution. In some embodiments, the first dilution is diluted by about 1-fold to about 100-fold. In some embodiments, the first dilution is diluted by about 1-fold to about 2-fold, about 1-fold to about 5-fold, about 1-fold to about 10-fold, about 1-fold to about 20-fold, about 1-fold to about 30-fold, about 1-fold to about 40-fold, about 1-fold to about 50-fold, about 1-fold to about 60-fold, about 1-fold to about 70-fold, about 1-fold to about 80-fold, about 1-fold to about 100-fold, about 2-fold to about 5-fold, about 2-fold to about 10-fold, about 2-fold to about 20-fold, about 2-fold to about 30-fold, about 2-fold to about 40-fold, about 2-fold to about 50-fold, about 2-fold to about 60-fold, about 2-fold to about 70-fold, about 2-fold to about 80-fold, about 2-fold to about 100-fold, about 5-fold to about 10-fold, about 5-fold to about 20-fold, about 5-fold to about 30-fold, about 5-fold to about 40-fold, about 5-fold to about 50-fold, about 5-fold to about 60-fold, about 5-fold to about 70-fold, about 5-fold to about 80-fold, about 5-fold to about 100-fold, about 10-fold to about 20-fold, about 10-fold to about 30-fold, about 10-fold to about 40-fold, about 10-fold to about 50-fold, about 10-fold to about 60-fold, about 10-fold to about 70-fold, about 10-fold to about 80-fold, about 10-fold to about 100-fold, about 20-fold to about 30-fold, about 20-fold to about 40-fold, about 20-fold to about 50-fold, about 20-fold to about 60-fold, about 20-fold to about 70-fold, about 20-fold to about 80-fold, about 20-fold to about 100-fold, about 30-fold to about 40-fold, about 30-fold to about 50-fold, about 30-fold to about 60-fold, about 30-fold to about 70-fold, about 30-fold to about 80-fold, about 30-fold to about 100-fold, about 40-fold to about 50-fold, about 40-fold to about 60-fold, about 40-fold to about 70-fold, about 40-fold to about 80-fold, about 40-fold to about 100-fold, about 50-fold to about 60-fold, about 50-fold to about 70-fold, about 50-fold to about 80-fold, about 50-fold to about 100-fold, about 60-fold to about 70-fold, about 60-fold to about 80-fold, about 60-fold to about 100-fold, about 70-fold to about 80-fold, about 70-fold to about 100-fold, or about 80-fold to about 100-fold. In some embodiments, the first dilution is diluted by about 1-fold, about 2-fold, about 5-fold, about 10-fold, about 20-fold, about 30-fold, about 40-fold, about 50-fold, about 60-fold, about 70-fold, about 80-fold, or about 100-fold. In some embodiments, the first dilution is diluted by at least about 1-fold, about 2-fold, about 5-fold, about 10-fold, about 20-fold, about 30-fold, about 40-fold, about 50-fold, about 60-fold, about 70-fold, or about 80-fold. In some embodiments, the first dilution is diluted by at most about 2-fold, about 5-fold, about 10-fold, about 20-fold, about 30-fold, about 40-fold, about 50-fold, about 60-fold, about 70-fold, about 80-fold, about 100-fold, or any numerical numbers in between the aforementioned dilution factors.

Further, as depicted in FIG. 2, after filtration/diafiltration/ultrafiltration process to remove at least a portion of the plurality of molecules that inhibits capping reaction of adding cap structures to uncapped RNA molecules, an optional step of washing the plurality of uncapped RNA molecules retained by the filter. In some embodiments, purified water without any RNAase or any other suitable washer may be used to perform this optional step. In some embodiments, this optional washing step may re-concentrate the plurality of uncapped RNA molecules.

In some preferred embodiments, the method for producing at least one capped ribonucleic acid (RNA) molecule, comprises:
providing a plurality of uncapped RNA molecules, obtained via an in vitro transcription (IVT) reaction, in a first solution, wherein said first solution comprises reagents for said IVT reaction;
removing a plurality of molecules that has a molecular weight of at most about 1000 kDa in the first solution to form a second solution, so that a post-transcriptional capping efficiency of at least 75% is achieved wherein the removing of the plurality of molecules is done by diafiltration or TFF;
contacting the second solution with a plurality of capping enzyme molecules; and
adding a cap structure to a 5′ end of an uncapped RNA molecule to form the at least one capped RNA molecule.

The method prevents the by-products and reagents of the IVT reaction to interfere with the capping process. In a preferred embodiment, the removal of the molecules from the first solution is the unique intermediate step between the IVT reaction and capping. This method simplifies the protocol of RNA processing after the IVT reaction and prior to the capping reaction, while providing high capping efficiencies and is as a result time- and cost-effective compared with state-of-the-art mRNA transcription and capping methods. The solution provided by the method disclosed herein does not require intermediate purification steps of RNA before performing the enzymatic capping and simplify therefore the manufacturing process while providing high yields of in vitro transcribed and capped RNA.

In some embodiments, the removal of the plurality of molecules that inhibits or interferes with capping reaction increases the capping efficiency in the second solution or allows capping reaction to achieve substantially similar capping efficiency compared to using conventional RNA purification methods. In some embodiments, the capping reaction is initiated by contacting the second solution with a plurality of capping enzymes and other reagents to form at least one capped RNA molecule or at least one capped mRNA molecule. In some embodiments, the filtering or dialyzing of the first solution increases the capping efficiency of the second solution to at least 50%, at least 55%, at least 60%, at least 65%, at least 70%, at least 75%, at least 77%, at least 80%, at least 85%, at least 87%, at least 90%, at least 95%, at least 96%, at least 97%, at least 98%, at least 99%, at least 99.5%, at least 99.9%, or any percentages in between the aforementioned percentages. In some embodiments, the filtering or dialyzing of the first solution allows capping reaction to occur at an efficiency of at least about 85%, 87%, 90%, 92%, 95%, 96%, 97%, 98%, 99%, 99.5%, 99.9%, or any percentages in between the aforementioned percentages.

In some embodiments, the capping efficiency may be determined by dividing the amount of uncapped RNA molecules by the amount of capped RNA molecules for obtaining a ratio of uncapped RNA/capped RNA. For example, but not meant to be limiting, liquid chromatography coupled with UV absorbance measurement and mass spectrometry (LC-UV-MS) may be used to assess capped or uncapped RNA molecule concentrations. In some embodiments, the concentrations of the uncapped RNA molecules and capped RNA molecules are calculated based on the absorbance readings of the eluted molecules as identified by in-line mass spectrometry. The capping efficiency is calculated based on the calculated concentrations. In some embodiments, the capping efficiency is calculated directly based on the absorbance readings of the capped RNA molecules and uncapped RNA molecules.

In some embodiments, the filtering or dialyzing of the first solution to obtain a second solution containing a plurality of uncapped RNA molecules and reduced level of molecules that inhibits capping reaction allows capping of the uncapped RNA molecules to commence and carry on. After the capping reaction, the ratio of uncapped RNA/capped RNA is at most 1.0, at most 0.8, at most 0.6, at most 0.4, at most 0.2, at most 0.1, at most 0.09, at most 0.08, at most 0.07, at most 0.06, at most 0.05, at most 0.04, at most 0.03, at most 0.02, at most 0.01, at most 0.001, or at most 0.0001. In some embodiments, the ratio between a plurality of uncapped RNA molecules and the plurality of capped RNA molecules is between about 0.001 to about 10. In some embodiments, the ratio between a plurality of uncapped RNA molecules and the plurality of capped RNA molecules is between about 0.001 to about 0.002, about 0.001 to about 0.005, about 0.001 to about 0.01, about 0.001 to about 0.02, about 0.001 to about 0.05, about 0.001 to about 0.1, about 0.001 to about 0.2, about 0.001 to about 0.5, about 0.001 to about 1, about 0.001 to about 5, about 0.001 to about 10, about 0.002 to about 0.005, about 0.002 to about 0.01, about 0.002 to about 0.02, about 0.002 to about 0.05, about 0.002 to about 0.1, about 0.002 to about 0.2, about 0.002 to about 0.5, about 0.002 to about 1, about 0.002 to about 5, about 0.002 to about 10, about 0.005 to about 0.01, about 0.005 to about 0.02, about 0.005 to about 0.05, about 0.005 to about 0.1, about 0.005 to about 0.2, about 0.005 to about 0.5, about 0.005 to about 1, about 0.005 to about 5, about 0.005 to about 10, about 0.01 to about 0.02, about 0.01 to about 0.05, about 0.01 to about 0.1, about 0.01 to about 0.2, about 0.01 to about 0.5, about 0.01 to about 1, about 0.01 to about 5, about 0.01 to about 10, about 0.02 to about 0.05, about 0.02 to about 0.1, about 0.02 to about 0.2, about 0.02 to about 0.5, about 0.02 to about 1, about 0.02 to about 5, about 0.02 to about 10, about 0.05 to about 0.1, about 0.05 to about 0.2, about 0.05 to about 0.5, about 0.05 to about 1, about 0.05 to about 5, about 0.05 to about 10, about 0.1 to about 0.2, about 0.1 to about 0.5, about 0.1 to about 1, about 0.1 to about 5, about 0.1 to about 10, about 0.2 to about 0.5, about 0.2 to about 1, about 0.2 to about 5, about 0.2 to about 10, about 0.5 to about 1, about 0.5 to about 5, about 0.5 to about 10, about 1 to about 5, about 1 to about 10, or about 5 to about 10. In some embodiments, the ratio between a plurality of uncapped RNA molecules and the plurality of capped RNA molecules is about 0.001, about 0.002, about 0.005, about 0.01, about 0.02, about 0.05, about 0.1, about 0.2, about 0.5, about 1, about 5, or about 10. In some embodiments, the ratio between a plurality of uncapped RNA molecules and the plurality of capped RNA molecules is at least about 0.001, about 0.002, about 0.005, about 0.01, about 0.02, about 0.05, about 0.1, about 0.2, about 0.5, about 1, or about 5. In some embodiments, the ratio between a plurality of uncapped RNA molecules and the plurality of capped RNA molecules is at most about 0.002, about 0.005, about 0.01, about 0.02, about 0.05, about 0.1, about 0.2, about 0.5, about 1, about 5, or about 10.

In some embodiments, during the step of filtering or dialyzing the first solution to form the second solution, no additional purification of the uncapped RNA molecules is needed. For example, the capping reaction can occur without inhibition by the method described herein without the need of utilizing chromatography or any other purification methods that are utilized to increase RNA capping reaction efficiency by current industry standards.

For filtration/diafiltration/ultrafiltration process, in some embodiments, the first solution goes through the process in the same container as the IVT RNA synthesis reaction. In some embodiments, the first solution is transferred to a different container to carry out the filtration process subsequent to the completion of the IVT RNA synthesis reaction. In some embodiments, filters (e.g., membrane filters) with certain pore sizes as described herein are used to separate molecules based on their sizes. The pore sizes of the filters are selected to remove the plurality of the molecules that inhibits the capping reaction of the uncapped RNA molecules. Also, the pore sizes of the filters are selected to retain the uncapped RNA molecules. In some cases, the filters are polymeric filters (e.g., polymeric membranes). The materials of the filters (e.g., membranes) may be any suitable materials that can be used in the filtration process. In some embodiments, a positive pressure is applied to the first solution against the filter during the filtration process to remove the plurality of molecules that inhibits capping reaction of the uncapped RNA molecules. In some embodiments, at least about 5%, 10%, 20%, 30%, 40%, 50%, 60%, 70%, 80%, 85%, 90%, 95%, 99% or approaching 100% of the plurality of the molecules that inhibits the capping reaction is removed. In some embodiments, direct flow filtration (DFF) process utilizing at least one pumping or centrifugal device is used to collect retentate, i.e., the second solution that comprises a plurality of uncapped RNA molecules and comprises a sufficiently reduced level of a plurality of molecules that inhibits capping reaction. In other embodiments, tangential flow filtration (TFF) process is utilized to collect the retentate as described herein. During TFF process, the first solution is passed parallel to a filtering membrane rather than being pushed through the filter perpendicularly.

In some embodiments, diafiltration process is utilized to collect retentate as described above. In some embodiments, the diafiltration process is a continuous diafiltration process. The diafiltration solution (water or any other suitable buffer) is added to a container holding the first solution at the same rate as filtrate (e.g., the solution containing a plurality of molecules that inhibits capping reaction) is generated. In this way, the volume in the container remains constant, but the smaller molecules (e.g., the plurality of molecules that inhibits capping reaction and other salts; having smaller molecular weight and size than uncapped RNA molecules) that can freely permeate through the filter are washed away. In some embodiments, the diafiltration process is discontinuous diafiltration. In some cases, at least about 5%, 10%, 20%, 30%, 40%, 50%, 60%, 70%, 80%, 85%, 90%, 95%, 99%, or approaching 100% by weight of the plurality of the molecules that inhibits the capping reaction is removed.

For dialysis process, in some embodiments, a dialysis device is provided to hold the IVT reaction mixture, i.e., the first solution. The dialysis device may be made of a filter capable of retaining the uncapped RNA molecules inside the dialysis device and letting the plurality of molecules that inhibits the capping reaction diffuse out from the dialysis device. In some embodiments, the filter comprises a molecule weight cut-off of at most about 1 kDa to about 1000 kDa. In some embodiments, the filter comprises a molecule weight cut-off of at most about 3 kDa to about 200 kDa. In some embodiments, the filter comprises a molecule weight cut-off of at most about 5 kDa to about 400 kDa. In some embodiments, the filter comprises a MWCO of at most about 10 kDa to about 750 kDa. In some embodiments, the filter comprises a MWCO of at least about 3 kDa, 5 kDa, 10 kDa, or 30 kDa. In some embodiments, the filter comprises a MWCO of at most about 200 kDa, 400 kDa, 500 kDa, 600 kDa, 750 kDa, or 800 kDa.

The dialysis step serves to remove at least a portion of the plurality of molecules that inhibits capping reaction. The dialysis process may comprise changing out dialysis buffer and adding fresh dialysis buffer to the IVT reaction mixture. This changing and adding cycle may be repeated several times until the plurality of molecules that inhibits capping reaction is removed to a level that no longer inhibits capping reaction. In some embodiments, the dialysis process does not require repeating the changing and adding cycle because adding dialysis buffer to the IVT reaction mixture one time is sufficient to remove the plurality of molecules that inhibits capping reaction to the level that no longer inhibits capping reaction.

In some embodiments, the first solution (i.e., the reaction mixture from the IVT RNA synthesis process) is not diluted prior to go through the filtration process to remove at least a portion of the plurality of the molecules that inhibits capping reaction. In some other embodiments, the first solution is not diluted prior to go through a dialysis process to reduce the concentration of the plurality of the molecules that inhibits capping reaction. In some embodiments, the dialysis device may be in any shape, such as a tubular shape. The dialysis device containing the first solution is submerged in a suitable dialysis buffer (e.g., water) to allow the plurality of molecules that inhibits the capping reaction to diffuse out into the dialysis buffer. The dialysis buffer might be stirred by a stirring device to aid the diffusion of the plurality of molecules that inhibits the capping reaction. In some embodiments, the dialysis buffer might be removed and replaced with fresh dialysis buffer when an equilibrium of the concentrations of the plurality of the molecules between the IVT reaction mixture contained in the dialysis device and the dialysis buffer is reached. The dialysis buffer might be replaced several times until a concentration of the plurality of the molecules that inhibits capping reaction is reduced to a suitable level that the efficiency of the capping reaction is increased by at least 50%, 55%, 60%, 65%, 70%, 75%, 80%, 85%, 90%, 95%, 99%, approaching 100% or any percentages in between the aforementioned percentages. In some embodiments, electricity might be used to aid the dialysis process.

In some embodiments, the first solution may be diluted in the same container as where the IVT and the capping reaction occurs. For example, additional solution may be added to the first solution in the same container to form a second (diluted) solution. In some embodiments, the first solution may be diluted by mixing a portion of the first solution with additional solution in a second container to form the second (diluted) solution. In some embodiments, any solution that is inert and does not interfere with capping reaction of the uncapped RNA or mRNA molecules may be used. In some embodiments, the diluting solution is water. In some embodiments, the second solution may be further purified (e.g., by chromatography) or concentrated (e.g., by filtration or ultrafiltration) prior to the capping reaction. In some embodiments, the volume of the second solution may be decreased prior to the capping reaction (e.g., by filtration or ultrafiltration). In some embodiments, the dilution of the first solution to form the second solution does not create excess volume and the capping reaction may be initiated directly in the second solution. In some embodiments, the capped RNA molecules or the capped mRNA molecules may then be purified from the second solution and further formulated (e.g., in a pharmaceutical composition described herein). In some embodiments, additional reactions may be carried out in the container after the capping reaction, where the capped RNA molecules function as template for synthesizing the compounds or biomolecules described herein.

In some embodiments, the method further comprises purifying or filtering a solution after the second solution undergoes capping reaction as shown in both FIGS. 1 and 2. The solution contains a plurality of capped RNA molecules. The plurality of capped RNA molecules is purified or filtered to remove unwanted substances and prepared for any other suitable downstream reactions described herein for producing pharmaceutical compositions. In some embodiments, the plurality of capped RNA molecules is not further purified or filtrated.

In some embodiments, the method comprises first synthesizing the uncapped RNA molecules. For example, the uncapped RNA molecules may be synthesized from in vitro transcription (IVT). In some embodiments, the IVT reaction may be carried out in any one of the containers of the system described herein. In some embodiments, the IVT reaction occurs in a continuous reactor. In some embodiments, the IVT reaction occurs in a batch reactor. In some embodiments, the IVT reaction occurs in a semi-continuous/simulated continuous mode, wherein the systems described herein comprises at least one batch reactor and at least one continuous reactor. In some embodiments, the IVT reaction may be terminated by inactivating RNA polymerase in the solution. RNA polymerase may be inactivated by heating, cooling, addition of chelator (e.g., 8-hydroxyquinoline, carboplatin, EDTA, EGTA, hyxadecylpyridinum bromide, or sodium tartrate), or a combination thereof. Non-limiting examples of RNA polymerase that may be used to synthesize the uncapped RNA molecules via IVT may include a T7 RNA polymerase, a T3 RNA polymerase, a SP6 RNA polymerase, a RNA polymerase I, a RNA polymerase II, a RNA polymerase III, a RNA polymerase IV, a RNA polymerase V, and a single subunit RNA polymerase. In some embodiments, the polymerases is T3 RNA polymerase, T7 RNA polymerase, or SP6 RNA polymerase.

An IVT reaction typically comprises nucleotide triphosphates (NTPs), a Rnase inhibitor and a DNA-dependent RNA polymerase in a transcription buffer. The NTPs may be naturally occurring NTPs and/or modified NTPs. The DNA-dependent RNA polymerase may be selected from but is not limited to T7 RNA polymerase, T3 RNA polymerase, SP6 RNA polymerase or mutant polymerases thereof.

In some embodiments, the method comprises contacting the second solution (with the plurality of molecules that inhibits capping reaction removed by the membrane filter), with at least one capping enzyme and other reagents (e.g., enzyme substrates, buffering agents, magnesium salts) for initiating the capping reaction in the second solution. Non-limiting examples of capping enzyme include Cap-specific mRNA (nucleoside-2′-O—)-methyltransferase, Vaccinia capping enzyme (VCE), Bluetongue Virus capping enzyme, Chlorella Virus capping enzyme, S. cerevisiae capping enzyme, Mimivirus capping enzyme, African swine fever virus capping enzyme, or Avian Reovirus capping enzyme. Non-limiting examples of other reagents may include Cap-specific mRNA (nucleoside-2′-O—)-methyltransferase, 2′-O-Methyltransferase, a magnesium salt, guanosine-5′-triphosphate, S-adenosylmethionine, buffering agents, RNase inhibitor. Non-limiting examples of capping structure include GpppN, m7GpppN (Cap 0), m7Gpppm6A, m7Gpppm1A, m7GpppNm (Cap 1), m2,7GpppNm, m2,2,7GpppNm, m7Gpppm6Am, m7Gpppm1Am, m7GpppNmpNm (Cap 2), m7GpppNmpNmpNm (Cap 3), m7GpppNmpNmpNmpNm (Cap 4), where N stands for any nucleotide, A for adenosine, G for guanosine, m for a methyl group and p for a phosphate group. In some embodiments, the capping structure comprises chemically modified nucleotide. In some embodiments, the capping reaction described herein yields a majority of one species of capped structure (e.g., Cap 1. In some embodiments, the capping reaction described herein yields other minor cap structures such as Cap 0, Cap 2, or other.

In some embodiments, the first solution comprises uncapped RNA molecules. In some embodiments, the uncapped RNA molecules may include long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Trans amplifying mRNA, RNA oligonucleotides, antisense oligonucleotides, small interfering RNA (SIRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), transcription start site-associated (TSSa-) RNAs, upstream antisense (ua) RNAs, and promoter upstream transcripts (PROMPTs). In some embodiments, the uncapped RNA molecules comprise at least one chemical modification comprising backbone modification, sugar modification, or base modification. In this context, a modified RNA molecule comprises nucleotide modifications, e.g. backbone modifications, sugar modifications or base modifications. A sugar modification in connection with the present disclosure is a chemical modification of the sugar of the nucleotides of the RNA molecule. Furthermore, a base modification in connection with the present disclosure is a chemical modification of the base moiety of the nucleotides of the RNA molecule. In this context, nucleotide modifications are selected from nucleotide modifications that are applicable for transcription and/or translation. In further embodiments, the modified RNA comprises nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

### Biomolecule Production

In some cases of the present disclosure, the systems and methods described herein are designed to accommodate a reaction/process or part of a reaction/process taking place in the system. In some cases, the reaction relates to processing a plurality of uncapped RNA molecules so that a capping reaction of adding a cap structure to an uncapped RNA molecule can carry one without inhibition. In some cases, the reaction relates to in vitro transcription (IVT) of RNA from a DNA template with ensuing post-transcriptional reactions, such as enzymatic capping and/or poly (A)-tail addition. In some cases, the reaction pertains to in vitro (cell-free) translation of RNA to protein. In some cases, the reaction pertains to a combination of both processes, i.e., from DNA to RNA through transcription and from RNA to protein through translation.

In some cases, the in vitro transcription relates to a process in which RNA is synthesized in a cell-free system (in vitro). In some cases, cloning vectors DNA, particularly plasmid DNA vectors are applied as templates for the generation of RNA transcripts following linearization of circular plasmid DNA molecule. These cloning vectors are generally designated as transcription vector. RNA may be obtained by DNA dependent in vitro transcription of an appropriate DNA template. A promoter for controlling RNA in vitro transcription may be any promoter for any DNA dependent RNA polymerase. In some embodiments, a viral RNA polymerase binds a viral promoter and is at least one promoter selected from the list consisting of T7, T3, T7lac, SP6, pL, pR, CMV, SV40, and CaMV35S. Alternately or in combination, the nucleic acid fragment comprising promoter sequence comprises a bacterial promoter. In some cases, a bacterial RNA polymerase binds a bacterial promoter and is at least one promoter selected from the list consisting of araBAD, trp, lac, and Ptac. In some cases, the nucleic acid fragment comprising promoter sequence comprises a eukaryotic promoter. In some cases, the eukaryotic RNA polymerase binds a eukaryotic promoter and is at least one promoter selected from the list consisting of EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. In some cases, the eukaryotic promoter is at least one promoter selected from the list consisting of an RNA pol I promoter, an RNA pol II promoter and an RNA pol III promoter.

In some cases, the DNA dependent RNA polymerases comprise at least one of a T7 RNA polymerase, a T3 RNA polymerase, a SP6 RNA polymerase, a RNA polymerase I, a RNA polymerase II, a RNA polymerase III, a RNA polymerase IV, a RNA polymerase V, and a single subunit RNA polymerase. The DNA template for in vitro RNA transcription may be obtained by cloning of a nucleic acid, in particular cDNA corresponding to the respective RNA to be in vitro transcribed, and introducing it into an appropriate vector for RNA in vitro transcription, for example in circular plasmid DNA which is introduced in a host such as a bacterium. The cDNA may be obtained by reverse transcription of mRNA, chemical synthesis or by amplification (for example, polymerase chain reaction). Moreover, the DNA template for in vitro RNA synthesis may also be obtained by gene synthesis.

In some cases, the DNA template relates to a DNA molecule comprising a nucleic acid sequence encoding the RNA sequence. The template DNA is used as a template for RNA in vitro transcription in order to produce the RNA encoded by the template DNA. Therefore, the template DNA comprises all elements necessary for RNA in vitro transcription, particularly a promoter element for binding of a DNA-dependent RNA polymerase as e.g. T3, T7 and SP6 RNA polymerases 5′ of the DNA sequence encoding the target RNA sequence. The poly (A) tail may be either encoded into the DNA template or added enzymatically to RNA in a separate step after in vitro transcription. In some cases, the template DNA comprises primer binding sites 5′ and/or 3′ of the DNA sequence encoding the target RNA sequence to determine the identity of the DNA sequence encoding the target RNA sequence e.g. by PCR or DNA sequencing. In some cases, the DNA template comprises a 5′ UTR or a 3′ UTR. In some cases, the DNA template comprises a DNA vector, such as a plasmid DNA, which comprises a nucleic acid sequence encoding the RNA sequence. In some cases, the DNA template comprises a linear or a circular DNA molecule.

In some cases of the present disclosure, a DNA template encodes a different RNA molecule species. In some cases the DNA template contains a sub-genomic promoter and a large ORF encoding for non-structural proteins which, following delivery of the biopharmaceutical into the cytosol, are transcribed in four functional components (nsP1, nsP2, nsP3, and nsp4) by the encoded RNA-dependent RNA polymerase (RDRP). RDRP than produces a negative-sense copy of the genome which serves as a template for two positive-strand RNA molecules: the genomic mRNA and a shorter sub-genomic mRNA. This sub-genomic mRNA is transcribed at very high levels, allowing the amplification of mRNA encoding the antigen of choice. A different RNA molecule species may encode an antigen of different serotypes or strains of a pathogen, a different allergen, a different autoimmune antigen, a different antigen of a pathogen, different adjuvant proteins, a different isoform or variant of a cancer or tumor antigen, a different tumor antigen of one patient, one antibody among a group of antibodies which target different epitopes of a protein or of a group of proteins, different proteins of a metabolic pathway, a single protein among a group of proteins which are defect in a subject, or a different isoform of a protein for molecular therapy.

In some embodiments, the RNA molecules capped by the method described herein comprises a non-coding region of a peptide or protein. In some embodiments, the RNA molecules capped by the method described herein comprises a coding region of a peptide or protein. In such case, the capped RNA molecules serve as a template for peptide or protein synthesis. For example, the capped RNA molecules may be further formulated into a composition or a pharmaceutical composition to be administered to a subject, where the synthesis of the peptide or protein occurs in vivo. In some embodiments, the peptide or protein may be synthesized directly from the capped RNA molecules either in the same or different container of the system. The in vitro synthesized peptide or protein may then be formulated into a composition or a pharmaceutical composition. In some embodiments, the peptide or protein encoded by the capped RNA molecules may be prophylactic. In some embodiments, the peptide or protein encoded by the capped RNA molecules may be therapeutic. In some embodiments, the peptide or protein encoded by the capped RNA molecules comprises a molecular weight (MW) of about 1 kDa to about 1,000 kDa. In some embodiments, the peptide or protein encoded by the capped RNA molecules comprises a molecular weight (MW) of about 10 kDa to about 20 kDa, about 10 kDa to about 50 kDa, about 10 kDa to about 100 kDa, about 10 kDa to about 200 kDa, about 10 kDa to about 300 kDa, about 10 kDa to about 400 kDa, about 10 kDa to about 500 kDa, about 10 kDa to about 600 kDa, about 10 kDa to about 700 kDa, about 10 kDa to about 800 kDa, about 10 kDa to about 1,000 kDa, about 20 kDa to about 50 kDa, about 20 kDa to about 100 kDa, about 20 kDa to about 200 kDa, about 20 kDa to about 300 kDa, about 20 kDa to about 400 kDa, about 20 kDa to about 500 kDa, about 20 kDa to about 600 kDa, about 20 kDa to about 700 kDa, about 20 kDa to about 800 kDa, about 20 kDa to about 1,000 kDa, about 50 kDa to about 100 kDa, about 50 kDa to about 200 kDa, about 50 kDa to about 300 kDa, about 50 kDa to about 400 kDa, about 50 kDa to about 500 kDa, about 50 kDa to about 600 kDa, about 50 kDa to about 700 kDa, about 50 kDa to about 800 kDa, about 50 kDa to about 1,000 kDa, about 100 kDa to about 200 kDa, about 100 kDa to about 300 kDa, about 100 kDa to about 400 kDa, about 100 kDa to about 500 kDa, about 100 kDa to about 600 kDa, about 100 kDa to about 700 kDa, about 100 kDa to about 800 kDa, about 100 kDa to about 1,000 kDa, about 200 kDa to about 300 kDa, about 200 kDa to about 400 kDa, about 200 kDa to about 500 kDa, about 200 kDa to about 600 kDa, about 200 kDa to about 700 kDa, about 200 kDa to about 800 kDa, about 200 kDa to about 1,000 kDa, about 300 kDa to about 400 kDa, about 300 kDa to about 500 kDa, about 300 kDa to about 600 kDa, about 300 kDa to about 700 kDa, about 300 kDa to about 800 kDa, about 300 kDa to about 1,000 kDa, about 400 kDa to about 500 kDa, about 400 kDa to about 600 kDa, about 400 kDa to about 700 kDa, about 400 kDa to about 800 kDa, about 400 kDa to about 1,000 kDa, about 500 kDa to about 600 kDa, about 500 kDa to about 700 kDa, about 500 kDa to about 800 kDa, about 500 kDa to about 1,000 kDa, about 600 kDa to about 700 kDa, about 600 kDa to about 800 kDa, about 600 kDa to about 1,000 kDa, about 700 kDa to about 800 kDa, about 700 kDa to about 1,000 kDa, or about 800 kDa to about 1,000 kDa. In some embodiments, the peptide or protein encoded by the capped RNA molecules comprises a molecular weight (MW) of about 10 kDa, about 20 kDa, about 50 kDa, about 100 kDa, about 200 kDa, about 300 kDa, about 400 kDa, about 500 kDa, about 600 kDa, about 700 kDa, about 800 kDa, or about 1,000 kDa. In some embodiments, the peptide or protein encoded by the capped RNA molecules comprises a molecular weight (MW) of at least about 10 kDa, about 20 kDa, about 50 kDa, about 100 kDa, about 200 kDa, about 300 kDa, about 400 kDa, about 500 kDa, about 600 kDa, about 700 kDa, or about 800 kDa. In some embodiments, the peptide or protein encoded by the capped RNA molecules comprises a molecular weight (MW) of at most about 20 kDa, about 50 kDa, about 100 kDa, about 200 kDa, about 300 kDa, about 400 kDa, about 500 kDa, about 600 kDa, about 700 kDa, about 800 kDa, or about 1,000 kDa.

In some embodiments, the peptide or protein encoded by the capped RNA molecules comprises a molecular weight (MW) at most about 10 kDa to about 1,000 kDa. In some embodiments, the peptide or protein encoded by the capped RNA molecules comprises a molecular weight (MW) at most about 10 kDa to about 20 kDa, about 10 kDa to about 50 kDa, about 10 kDa to about 100 kDa, about 10 kDa to about 200 kDa, about 10 kDa to about 300 kDa, about 10 kDa to about 400 kDa, about 10 kDa to about 500 kDa, about 10 kDa to about 600 kDa, about 10 kDa to about 700 kDa, about 10 kDa to about 800 kDa, about 10 kDa to about 1,000 kDa, about 20 kDa to about 50 kDa, about 20 kDa to about 100 kDa, about 20 kDa to about 200 kDa, about 20 kDa to about 300 kDa, about 20 kDa to about 400 kDa, about 20 kDa to about 500 kDa, about 20 kDa to about 600 kDa, about 20 kDa to about 700 kDa, about 20 kDa to about 800 kDa, about 20 kDa to about 1,000 kDa, about 50 kDa to about 100 kDa, about 50 kDa to about 200 kDa, about 50 kDa to about 300 kDa, about 50 kDa to about 400 kDa, about 50 kDa to about 500 kDa, about 50 kDa to about 600 kDa, about 50 kDa to about 700 kDa, about 50 kDa to about 800 kDa, about 50 kDa to about 1,000 kDa, about 100 kDa to about 200 kDa, about 100 kDa to about 300 kDa, about 100 kDa to about 400 kDa, about 100 kDa to about 500 kDa, about 100 kDa to about 600 kDa, about 100 kDa to about 700 kDa, about 100 kDa to about 800 kDa, about 100 kDa to about 1,000 kDa, about 200 kDa to about 300 kDa, about 200 kDa to about 400 kDa, about 200 kDa to about 500 kDa, about 200 kDa to about 600 kDa, about 200 kDa to about 700 kDa, about 200 kDa to about 800 kDa, about 200 kDa to about 1,000 kDa, about 300 kDa to about 400 kDa, about 300 kDa to about 500 kDa, about 300 kDa to about 600 kDa, about 300 kDa to about 700 kDa, about 300 kDa to about 800 kDa, about 300 kDa to about 1,000 kDa, about 400 kDa to about 500 kDa, about 400 kDa to about 600 kDa, about 400 kDa to about 700 kDa, about 400 kDa to about 800 kDa, about 400 kDa to about 1,000 kDa, about 500 kDa to about 600 kDa, about 500 kDa to about 700 kDa, about 500 kDa to about 800 kDa, about 500 kDa to about 1,000 kDa, about 600 kDa to about 700 kDa, about 600 kDa to about 800 kDa, about 600 kDa to about 1,000 kDa, about 700 kDa to about 800 kDa, about 700 kDa to about 1,000 kDa, or about 800 kDa to about 1,000 kDa. In some embodiments, the peptide or protein encoded by the capped RNA molecules comprises a molecular weight (MW) at most about 10 kDa, about 20 kDa, about 50 kDa, about 100 kDa, about 200 kDa, about 300 kDa, about 400 kDa, about 500 kDa, about 600 kDa, about 700 kDa, about 800 kDa, or about 1,000 kDa. In some embodiments, the peptide or protein encoded by the capped RNA molecules comprises a molecular weight (MW) at most at least about 10 kDa, about 20 kDa, about 50 kDa, about 100 kDa, about 200 kDa, about 300 kDa, about 400 kDa, about 500 kDa, about 600 kDa, about 700 kDa, or about 800 kDa. In some embodiments, the peptide or protein encoded by the capped RNA molecules comprises a molecular weight (MW) at most at most about 20 kDa, about 50 kDa, about 100 kDa, about 200 kDa, about 300 kDa, about 400 kDa, about 500 kDa, about 600 kDa, about 700 kDa, about 800 kDa, or about 1,000 kDa.

### Compositions

Described herein is a composition comprising an agent or a composition described herein (e.g., the uncapped RNA molecules after processing as described herein). In some embodiments, the composition comprises substances with a MW of 400 kDa or less at a concentration of less than 15 v/v %. In some embodiments, the substances comprise an MW of about 20 kDa, about 50 kDa, about 100 kDa, about 200 kDa, about 300 kDa, about 400 kDa, about 500 kDa, about 600 kDa, about 700 kDa, about 800 kDa, or about 1,000 kDa. In some embodiments, the substances comprise a concentration of less than 30 v/v %, less than 25 v/v %, less than 20 v/v %, less than 19 v/v %, less than 18 v/v %, less than 17 v/v %, less than 16 v/v %, less than 15 v/v %, less than 14 v/v %, less than 13 v/v %, less than 12 v/v %, less than 11 v/v %, less than 10 v/v %, less than 9 v/v %, less than 8 v/v %, less than 7 v/v %, less than 6 v/v %, less than 5 v/v %, less than 4 v/v %, less than 3 v/v %, less than 2 v/v %, or less than 1 v/v %.

In some embodiments, the composition comprises a plurality of 5′ uncapped RNA molecules, wherein said uncapped RNA molecules are obtained by means of an in vitro transcription reaction, said composition comprises reagents for in vitro transcription, and wherein the concentration of uncapped RNA in said composition is less than 20 mg/ml. In some embodiments, the concentration of the uncapped RNA in said composition comprises a range between about 0.01 mg/ml to about 20 mg/ml. In some embodiments, the concentration of the uncapped RNA in said composition comprises a range between about 0.01 mg/ml to about 0.02 mg/ml, about 0.01 mg/ml to about 0.05 mg/ml, about 0.01 mg/ml to about 0.1 mg/ml, about 0.01 mg/ml to about 0.2 mg/ml, about 0.01 mg/ml to about 0.5 mg/ml, about 0.01 mg/ml to about 1 mg/ml, about 0.01 mg/ml to about 2 mg/ml, about 0.01 mg/ml to about 5 mg/ml, about 0.01 mg/ml to about 10 mg/ml, about 0.01 mg/ml to about 20 mg/ml, about 0.02 mg/ml to about 0.05 mg/ml, about 0.02 mg/ml to about 0.1 mg/ml, about 0.02 mg/ml to about 0.2 mg/ml, about 0.02 mg/ml to about 0.5 mg/ml, about 0.02 mg/ml to about 1 mg/ml, about 0.02 mg/ml to about 2 mg/ml, about 0.02 mg/ml to about 5 mg/ml, about 0.02 mg/ml to about 10 mg/ml, about 0.02 mg/ml to about 20 mg/ml, about 0.05 mg/ml to about 0.1 mg/ml, about 0.05 mg/ml to about 0.2 mg/ml, about 0.05 mg/ml to about 0.5 mg/ml, about 0.05 mg/ml to about 1 mg/ml, about 0.05 mg/ml to about 2 mg/ml, about 0.05 mg/ml to about 5 mg/ml, about 0.05 mg/ml to about 10 mg/ml, about 0.05 mg/ml to about 20 mg/ml, about 0.1 mg/ml to about 0.2 mg/ml, about 0.1 mg/ml to about 0.5 mg/ml, about 0.1 mg/ml to about 1 mg/ml, about 0.1 mg/ml to about 2 mg/ml, about 0.1 mg/ml to about 5 mg/ml, about 0.1 mg/ml to about 10 mg/ml, about 0.1 mg/ml to about 20 mg/ml, about 0.2 mg/ml to about 0.5 mg/ml, about 0.2 mg/ml to about 1 mg/ml, about 0.2 mg/ml to about 2 mg/ml, about 0.2 mg/ml to about 5 mg/ml, about 0.2 mg/ml to about 10 mg/ml, about 0.2 mg/ml to about 20 mg/ml, about 0.5 mg/ml to about 1 mg/ml, about 0.5 mg/ml to about 2 mg/ml, about 0.5 mg/ml to about 5 mg/ml, about 0.5 mg/ml to about 10 mg/ml, about 0.5 mg/ml to about 20 mg/ml, about 1 mg/ml to about 2 mg/ml, about 1 mg/ml to about 5 mg/ml, about 1 mg/ml to about 10 mg/ml, about 1 mg/ml to about 20 mg/ml, about 2 mg/ml to about 5 mg/ml, about 2 mg/ml to about 10 mg/ml, about 2 mg/ml to about 20 mg/ml, about 5 mg/ml to about 10 mg/ml, about 5 mg/ml to about 20 mg/ml, or about 10 mg/ml to about 20 mg/ml. In some embodiments, the concentration of the uncapped RNA in said composition comprises a range between about 0.01 mg/ml, about 0.02 mg/ml, about 0.05 mg/ml, about 0.1 mg/ml, about 0.2 mg/ml, about 0.5 mg/ml, about 1 mg/ml, about 2 mg/ml, about 5 mg/ml, about 10 mg/ml, or about 20 mg/ml. In some embodiments, the concentration of the uncapped RNA in said composition comprises a range between at least about 0.01 mg/ml, about 0.02 mg/ml, about 0.05 mg/ml, about 0.1 mg/ml, about 0.2 mg/ml, about 0.5 mg/ml, about 1 mg/ml, about 2 mg/ml, about 5 mg/ml, or about 10 mg/ml. In some embodiments, the concentration of the uncapped RNA in said composition comprises a range between at most about 0.02 mg/ml, about 0.05 mg/ml, about 0.1 mg/ml, about 0.2 mg/ml, about 0.5 mg/ml, about 1 mg/ml, about 2 mg/ml, about 5 mg/ml, about 10 mg/ml, or about 20 mg/ml.

In some embodiments, the composition comprises a plurality of 5′-capped RNA molecules, wherein a capping reaction occurs post-transcriptionally and wherein a ratio between a plurality of uncapped RNA molecules and the plurality of capped RNA molecules is between 0.0001 and 1. In some embodiments, the composition comprises a plurality of 5′-capped RNA molecules, wherein a capping reaction occurs post-transcriptionally and wherein a ratio between a plurality of uncapped RNA molecules and the plurality of capped RNA molecules is between 0.01 and 0.5. In some embodiments, the composition comprises the capped RNA molecules or peptide encoded by the capped RNA molecules. In some embodiments, the capped RNA molecules or peptide encoded by the capped RNA molecules may encode an antigen for vaccine formulation. In some embodiments, the capped RNA molecules are further processed for formulation into a vaccine composition.

In some embodiments, the vaccine composition comprises at least one 5′ capped RNA molecule, where the 5′ capped RNA molecule is a 5′ capped mRNA molecule. In some embodiments, the at least one 5′ capped mRNA molecule can be encapsulated in a nanoparticle to form a pharmaceutical composition. The nanoparticle can comprise lipids, carbohydrates, polypeptides, polymers formed from one or more monomers, or any combination thereof (including molecular combinations of these substances). In some embodiments, the at least one 5′ capped RNA molecules is complexed with a charged polymer, e.g. by electrostatic interaction

In some embodiments, the pharmaceutical composition comprising the at least one 5′ capped mRNA molecule can be formulated with a charged lipid or an amino lipid. In some embodiments, the pharmaceutical composition comprising the at least one 5′ capped mRNA molecule can be formulated by complexing with lipids, liposomes, or lipoplexes. For example, the complexing can include contacting the at least one 5′ capped mRNA molecule with a PEG-lipid or a zwitterionic lipid comprising a headgroup, where the positive charge is located near the acyl chain region and the negative charge is located at the distal end of the head group. In some embodiments, the pharmaceutical composition comprising the at least one 5′ capped mRNA molecule can be formulated with a lipid bilayer carrier.

In some embodiments, the pharmaceutical composition comprising the at least one 5′ capped mRNA molecule comprises can be formulated with a natural or synthetic polymer. Non-limiting examples of such polymers can include chitosan or cyclodextrin. In some embodiments, the pharmaceutical composition comprising the at least one 5′ capped mRNA molecule comprises can be formulated in a polymeric formulation comprising polymer such polyethenes, polyethylene glycol (PEG), poly (I-lysine) (PLL), cationic lipopolymer, biodegradable cationic lipopolymer, polyethyleneimine (PEI), cross-linked branched poly (alkylene imines), a polyamine derivative, a modified poloxamer, a biodegradable polymer, elastic biodegradable polymer, acrylic polymers, amine-containing polymer, dextran polymer, dextran polymer derivative, or a combination thereof. In some embodiments, the pharmaceutical composition comprising the at least one 5′ capped mRNA molecule comprises can be formulated in a polyplex with one or more polymers commonly used in pharmaceutical formulation. In some embodiments, the polyplex comprises two or more cationic polymers such as poly (ethylene imine) (PEI). In some embodiments, the pharmaceutical composition comprising the at least one 5′ capped mRNA molecule comprises can be formulated as a nanoparticle using a combination of polymers, lipids, or other biodegradable agents. In some embodiments, the lipid nanoparticles may comprise a core of the 5′ capped mRNA described herein and a polymer shell. The polymer shell can be any of the polymers known in pharmaceutical formulation.

In some embodiments, the pharmaceutical composition comprises a pharmaceutically acceptable: carrier, excipient, or diluent. In some embodiments, the pharmaceutical composition described herein includes at least one additional active agent described herein. In some embodiments, the at least one additional active agent is a chemotherapeutic agent, cytotoxic agent, cytokine, growth-inhibitory agent, anti-hormonal agent, anti-angiogenic agent, or checkpoint inhibitor. In some embodiments, the at least one additional active agent is an adjuvant for increasing effectiveness of vaccination.

In practicing the methods of treatment or use provided herein, therapeutically effective amount of pharmaceutical composition described herein is administered to a mammal having a disease, disorder, or condition to be treated. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended-release formulations, pulsatile release formulations, multi-particulate formulations, and mixed immediate and controlled release formulations.

The pharmaceutical composition including a therapeutic agent may be manufactured in a conventional manner, such as, by way of example only, by means of conventional mixing, chaotic mixing, laminar mixing, dissolving, encapsulating or other processes.

The pharmaceutical composition may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these biomolecules having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or any numerical numbers in between the aforementioned dilution factors compared to a reference level.

The terms “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease.

The terms “RNA” or “RNA molecule” are used herein to generally refer to any type RNA. Non-limiting example of RNA includes long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Trans amplifying mRNA, RNA oligonucleotides, antisense oligonucleotides, small interfering RNA (SIRNA), small hairpin RNA (shRNA), antisense RNA (asRNA), CRISPR/Cas9 guide RNAs, riboswitches, immunostimulating RNA (iSRNA), ribozymes, aptamers, ribosomal RNA (rRNA), transfer RNA (tRNA), viral RNA (VRNA), retroviral RNA or replicon RNA, small nuclear RNA (snRNA), small nucleolar RNA (snoRNA), microRNA (miRNA), transcription start site-associated (TSSa-) RNAs, upstream antisense (ua) RNAs, promoter upstream transcripts (PROMPTs), or a combination thereof.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the systems and methods described herein and are not meant to be limiting in any way.

### Example 1. Capping Nucleic Acid Following Buffer Exchange

The capping efficiency of RNA transcribed in vitro was increased by diluting the solution containing the uncapped RNA molecules prior to the capping reaction. FIG. 1 and FIG. 2 illustrate non-limiting examples of the experiments for performing buffer exchange and dialysis for capping the in vitro transcribed (IVT) RNA. The solution containing the uncapped RNA molecules can be contacted with a filter comprising a kDa molecular weight cut-off (10 kDa MWCO). After removal of the solution by centrifugation, the retentate can be re-diluted in RNase-free water and the resulting solution again filtered through the 10 kDa MWCO filter. This process can be repeated one more time. Then, the retentate can be dissolved in RNase free water, and the uncapped RNA molecules can be contacted with capping enzymes and other capping reagents (GTP, capping buffer concentrate, S-adenosylmethionine, MgCl2) for initiating the capping reaction.

### Example 2. Method of Producing Capped RNA

### IVT Reaction

A linearized DNA template of a gene of interest was transcribed into mRNA using an IVT reaction. The IVT reaction comprised nucleotide triphosphates (NTPs), a RNase inhibitor and a DNA-dependent RNA polymerase in a transcription buffer. The IVT reaction was carried out at 37° C. for 1 to 3 hours.

### Pre-Treatment by Filtration Before Enzymatic Capping

After the completion of the IVT reaction, the IVT obtained mRNA was 3-fold diluted with nuclease-free water. In alternative embodiments, the mRNA is diluted 2 to 250-fold. The diluted mRNA samples were centrifuged at 5000 g in 10 kDa filter tubes (Pall Macrosep® Advance; MAP010C36). A second centrifugation followed at 14 000 g in 10 kDa filter tubes (Amicon Ultra-0,5; UFC501096). All centrifugation steps were performed at 4° C. for a sufficient time to recover a volume of retentate equivalent to the initial volume before dilution.

mRNA Capping

The recovered mRNA samples were heated at 65° C. for 5 minutes and then cooled on ice. The enzymatic capping reaction was carried out with a NEB® kit (New England BioLabs) according to the supplier protocol. Briefly, the NEB® kit components, namely, 10× capping buffer, GTP 10 mM, SAM (4 mM) and Vaccinia capping enzyme (10 U/μl) were added to the reaction unit, together with mRNA Cap 2′-O-Methyltransferase (50 U/μl) and a RNase inhibitor, not included in the kit. The capping reaction was carried out at 37° C. for 60 minutes.

Capping Efficiency and Analysis of Capped mRNA Species

The capped mRNA was incubated with RNase A and probes to protect the 5′ end from degradation. The capped mRNA was isolated and purified with magnetic beads. After elution, the small RNA fragments were analyzed by denaturing ion-paired reversed-phase high-performance liquid chromatography (IP-RP-HPLC) coupled with electrospray ionization mass spectrometry (ESI-MS) to determine the percentages of cap 1, cap 0, unmethylated cap and uncapped RNA in the sample.

Results: The capping efficiencies of the mRNA filtered directly after the IVT reaction and capped afterward are shown in Table 1. The results show that the enzymatic capping was achieved when the mRNA was filtered immediately after the IVT reaction. The capping efficiencies varied between 94 and 96%.

Concluding, mRNA can be very efficiently capped when the IVT reaction solution is filtered beforehand. The filtration step of the IVT solution is mandatory as the control condition (i.e., without any treatment beforehand) failed to be capped.

TABLE 1
Capping efficiency of mRNA capped after filtration

| | total | unmethylated | | |
|---|---|---|---|---|
| Conditions | capped % | cap % | cap 0% | cap 1% |
| Control: untreated | 0.0% | 0.0% | 0.0% | 0.0% |
| IVT RNA followed by | | | | |
| enzymatic capping | | | | |
| process | | | | |
| uncapped IVT RNA | 96.0% | 0.0% | 1.0% | 95.0% |
| with 10 kDa filter | 96.2% | 0.0% | 1.3% | 94.9% |
| followed by enzymatic | 95.9% | 0.0% | 1.3% | 94.6% |
| capping process | 94% | 4% | 0% | 91% |

While the foregoing disclosure has been described in some detail for purposes of clarity and understanding, it will be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

1. A method for producing at least one capped ribonucleic acid (RNA) molecule, comprising:
a) providing a plurality of uncapped RNA molecules in a first solution, wherein the plurality of uncapped RNA molecules is obtained via an in vitro transcription (IVT) reaction and wherein the first solution comprises reagents for the IVT reaction;
b) removing a plurality of molecules that has a molecular weight of at most about 1000 kDa in the first solution to form a second solution to achieve a post-transcriptional capping efficiency of at least 75%, wherein the removing of the plurality of molecules comprises filtration of the first solution;
c) contacting the second solution with a plurality of capping enzyme molecules; and
d) adding a cap structure to a 5′ end of an uncapped RNA molecule to form the at least one capped RNA molecule.

2. The method of claim 1, wherein the plurality of molecules has a molecular weight of at most about 500 kDa.

3. The method of claim 1, wherein the plurality of molecules has a molecular weight of at most about 10 kDa.

4. The method of claim 1, wherein the plurality of molecules has a molecular weight of at most about 3 kDa.

5. The method of claim 1, wherein the filtration of the first solution comprises contacting the first solution with a filter and wherein the filter comprises a nominal pore size measured in a molecular weight cut off (MWCO) of about 800 kDa, 600 kDa, 500 kDa, 400 kDa, 200 kDa, or 100 kDa.

6. The method of claim 5, wherein the filter comprises a nominal pore size measured in MWCO of about 50 kDa, 30 kDa, 10 kDa, 5 kDa, 3 kDa, or 1 kDa.

7. The method of claim 1, wherein the filtration is by continuous or discontinuous diafiltration.

8. The method of claim 1, wherein the filtration comprises tangential flow filtration.

9. The method of claim 1, wherein the removing the plurality of molecules comprises conducting a dialysis of the first solution in a suitable medium.

10. The method of claim 1, wherein the removing the plurality of molecules does not comprise an additional purification step.

11. The method of claim 1, wherein the plurality of uncapped RNA molecules is generated via an in vitro transcription (IVT) reaction.

12. The method of claim 1, wherein the plurality of capping enzyme is selected from the group consisting of Cap-specific mRNA (nucleoside-2′-O—)-methyltransferase, Vaccinia capping enzyme (VCE), Bluetongue Virus capping enzyme, Chlorella Virus capping enzyme, S. cerevisiae capping enzyme, Mimivirus capping enzyme, African swine fever virus capping enzyme, and Avian Reovirus capping enzyme.

13. The method of claim 1, wherein adding the cap structure to the 5′ end of the uncapped RNA molecule occurs at an efficiency of at least 80%.

14. The method of claim 1, wherein adding the cap structure to the 5′ end of the uncapped RNA molecule occurs at an efficiency of at least 95%.

15. The method of any of the claims 1 to 20, wherein adding the cap structure to the 5′ end of the uncapped RNA molecule occurs at an efficiency of at least 99%.

16. The method of claim 1, wherein a concentration of the plurality of molecules is reduced by at least 50%, 60%, 70%, 80%, 85%, 90%, 95%, 96%, 97%, 98%, or 99% in the second solution compared to a concentration of the plurality of molecules in the first solution.

17. The method of claim 1, wherein a concentration of the plurality of molecules is reduced by at least 99.9975% in the second solution compared to a concentration of the plurality of molecules in the first solution.

18. The method of claim 1, further comprising synthesizing a peptide or protein utilizing the at least one capped RNA molecule.

19. A pharmaceutical composition comprising a plurality of 5′ capped RNA molecules, wherein the RNA molecules are obtained by means of an in vitro transcription reaction and wherein said capping occurred post-transcriptionally, said composition comprises reagents for in vitro transcription, and wherein the concentration of RNA in said composition is less than 20 mg/ml.

20. A pharmaceutical composition comprising a plurality of 5′-capped and uncapped RNA molecules, the RNA molecules are obtained by means of an in vitro transcription reaction and wherein said capping occurred post-transcriptionally, wherein a ratio between the plurality of uncapped RNA molecules and the plurality of capped RNA molecules is between 0.0001 to 0.3.
